(19) United States
(12) Patent Application Publication
(10) Pub. No.: US 2014/0275973 A1
(43) Pub. Date: Sep. 18, 2014

(54) MRI COIL DRAPE AND METHOD OF USING

(71) Applicant: pro med instruments Gmbh, Freiburg i.Br (DE)

(72) Inventor: Matthias E. Schuele, Freiburg (DE)

(73) Assignee: pro med instruments Gmbh, Freiburg i.Br (DE)

(21) Appl. No.: 14/211,853

(22) Filed: Mar. 14, 2014

Related U.S. Application Data
(60) Provisional application No. 61/788,579, filed on Mar. 15, 2013.

(51) Int. Cl. A61B 19/08; A61B 5/055

(52) U.S. Cl. CPC A61B 19/081; A61B 5/0555
USPC 600/415

(57) ABSTRACT

A drape is provided for use in a surgical or diagnostic procedure using MRI and a patient stabilization device. The drape protects a coil portion of a coil. The drape further includes one or more attachment members to selectively attach and secure the drape to the stabilization device.

### PRIORITY

This application is a non-provisional application of U.S. Provisional Patent App. Ser. No. 61/788,579, entitled “MRI Flex Coil Drape and Method of Using,” filed Mar. 15, 2013, the disclosure of which is incorporated by reference herein.

### BACKGROUND

When using certain imaging modalities such as MRI, coils are used that closely surround a body part of interest. These coils emit a radiofrequency that causes hydrogen atom nuclei to change their spin orientation moving the atom nuclei from a high-energy to a low-energy state. The detected energy released by molecules during this energy state change provides the signal that is used in creating MRI images. In some MRIs, the coil used is a flex coil designed to be placed anywhere on the body. Flex coils can provide a range of adjustment given their flexible nature compared to other non-flexible coils. When used in surgery it may be necessary or desirable to protect a flex coil against direct contact with fluids etc.

While a variety of ways to protect MRI coils may have been made and used, it is believed that no one prior to the inventor(s) has made or used an invention as described herein.

### BRIEF DESCRIPTION OF THE DRAWINGS

It is believed the present invention will be better understood from the following description of certain examples taken in conjunction with the accompanying drawings, in which like reference numerals identify the same elements.

FIG. 1 depicts a perspective view of an example MRI flex coil separate from an example drape configured to be installed on the MRI flex coil.

FIG. 2 depicts a perspective view of the MRI flex coil and drape of FIG. 1, with the drape partially installed on the MRI flex coil.

FIG. 3 depicts another perspective view of the MRI flex coil and drape of FIG. 1, with the drape partially installed on the MRI flex coil.

FIG. 4 depicts a perspective view of the MRI flex coil and drape of FIG. 1, shown with the drape installed on the MRI flex coil, and shown with an example head fixation device in the form of a skull clamp.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

FIG. 1 illustrates flex coil (100) and drape (200) separate from one another. Flex coil (100) comprises receiving member (102), shaft (104), connector (106), and coil portion (108). Receiving member (102) is configured to connect flex coil (100) with an MRI machine and power source for signal processing and ultimate image generation. Shaft (104) can be of a variety of lengths and couples coil portion (108) with receiving member (102). Connector (106) attaches coil portion (108) with shaft (104). In the present example, coil portion (108) is flexible to accommodate the shape of a body part, such as a head. In other versions, coil portion (108) is not flexible. In the present example, coil portion (108) has a arcuate shape, but in other versions coil (100) can have other suitable shapes, including for example, a round shape having an opening in the center, a square shape having an opening in the center, or a rectangular shape having an opening in the center. Other suitable shapes for coil (100) and coil portion (108) will be apparent to those of ordinary skill in the art in view of the teachings herein. Various wiring and other electrical components of flex coil (100) may be located within these portions of flex coil (100) as those of ordinary skill in the art will understand in view of the teachings herein.

Drape (200) comprises first portion (202) and second portion (204). First portion (202) has a greater width than second portion (204) and is configured to cover coil portion (108) of flex coil (100). Second portion (204) is configured to cover portions of shaft (104) and connector (106) of flex coil (100). Drape (200) further comprises interior space (206) that is defined by first surface (208) and second surface (210) of drape (200). Interior space (206) comprises first space (212) that generally coincides with the interior of first portion (202). Interior space (206) further comprises second space (214) that generally coincides with the interior of second portion (204). First space (212) and second space (214) are in communication with one another. In other words, first space (212) and second space (214) connect to form a continuous interior space (206). Drape (200) further comprises first opening (216) near the end of first portion (202) and second opening (218) near the end of second portion (204).

Drape (200) further comprises first tie strap (220) and second tie strap (222). In the present example, first and second tie straps (220, 222) are affixed to opposing side portions of first surface (208) of first portion (202) of drape (200). In other versions, first and second tie straps (220, 222) are affixed to other surfaces of drape (200), as will be apparent to one with ordinary skill in the art in view of the teachings herein. First and second tie straps (220, 222) are affixed to first surface (208) at or near a central portion of first and second tie straps (220, 222), as shown in the illustrated version. Drape (200) also comprises third tie strap (224) that is affixed to first surface (208) of second portion (204) of drape (200). In other versions, third tie strap (224) is affixed to other surfaces of drape (200), as will be apparent to one with ordinary skill in the art in view of the teachings herein. Third tie strap (224) is affixed to first surface (208) at or near an end portion of third tie strap (224) as also shown in the illustrated version. In FIG. 1, the areas of tie straps (220, 222, 224) that are affixed to first surface (208) are shown with dark shading. Tie straps (220, 222, 224) can be affixed to first surface (208) by an adhesive, an ultrasonic bond, fusion, a mechanical fastening, etc. These and other ways to affix tie straps (220, 222, 224) to first surface (208) will be apparent to those of ordinary skill in the art in view of the teachings herein.

Drape (200) also comprises first and second fasteners (226, 228). In some versions first and second fasteners (226, 228) comprise tape. In some other versions, first and second fasteners (226, 228) comprise hook material that can selectively engage first surface (208) or a loop material that can be affixed to first surface (208). In the illustrated version, first and second fasteners (226, 228) are affixed to first surface (208) near the end of first portion (202) adjacent first opening (216) such that first and second fasteners (226, 228) extend from first portion (202). In other versions, first and second fasteners (226, 228) are affixed to other surfaces of drape (200), as will be apparent to one with ordinary skill in the art in view of the teachings herein. In FIG. 1, the areas of first and second fasteners (226, 228) that are affixed to first surface (208) are shown with dark shading. First and second fasteners (226, 228) can be affixed to first surface (208) by an adhesive, an ultrasonic bond, fusion, a mechanical fastening, etc. These and other ways to affix first and second fasteners (226, 228) to first surface (208) will be apparent to those of ordinary skill in the art in view of the teachings herein. For instance, instead of being affixed to first surface (208) in some versions one or both of first and second fasteners (226, 228) can instead be affixed to second surface (210).

FIG. 2 illustrates a first state during installation of drape (200) over flex coil (100). As shown, receiving member (102) is inserted into interior space (206) through first opening (216) and passes through first space (212) and second space (214) until receiving member (102) ultimately emerges from and extends past second opening (218). With this configuration, second portion (204) of drape (200) covers portions of connector (106) and shaft (104).

During this process, coil portion (108) is moved within interior space (206). Coil portion (108) is positioned within first space (212) of interior space (206) until leading edge (110) of coil portion (108) is adjacent to or contacts trailing edge (230) of drape (200). This is approximately where first portion (202) of drape (200) terminates and second portion (204) of drape (200) begins. In this position, first portion (202) of drape (200) fully encompasses coil portion (108) and extends past coil portion (108).

FIG. 3 illustrates a second state during installation of drape (200) over flex coil (100). As shown, upper section (232) of first portion (202) is folded over coil portion (108) to close first opening (216). Upper section (232) is folded onto first surface (208) or second surface (210). To hold upper section (232) in place, first and second fasteners (226, 228) attach upper section (232) to first portion (202) of drape (200). In some versions, first and second fasteners (226, 228) attach upper section (232) to a remaining section of first portion (202) along first surface (208) of drape (200). In some other versions, first and second fasteners (226, 228) attach upper section (232) to a remaining section of first portion (202) along second surface (210) of drape (200). By way of example, and not limitation, this configuration is shown in FIG. 3 with first and second fasteners (226, 228) shown in phantom as indicated by the dotted lines. This maintains the position of coil portion (108) relative to first portion (202) of drape (200). Accordingly, coil portion (108) is coupled with first portion (202) of drape (200) to prevent unintentional movement of coil portion (108) relative to first portion (202) of drape (200).

Also as shown in FIG. 3, third tie strap (224) is wrapped around connector (106) to close second opening (218). Third tie strap (224) can be tied to selectively hold second portion (204) closed. In some versions, third tie strap (224) has an adhesive backing such that it can be adhered to itself or second portion (204) that way. Still in other versions, third tie strap (224) can comprise a tape for adhering third tie strap (224) to second portion (204). Still yet in other versions, third tie strap (224) can comprise a hook material for selectively engaging first or second surfaces (208, 210), or for engaging a portion of third tie strap (224) itself. In view of the teachings herein, other ways to close second opening (218) using third tie strap (224) or another structure will be apparent to those of ordinary skill in the art. With first and second openings (216, 218) closed, coil portion (108) is fully covered and protected such that flex coil (100) is ready to be positioned for use as described further below.

FIG. 4 illustrates the flex coil (100) protected by drape (200) and positioned within the space defined by first and second arms (302, 304) of an example head fixation device in the form of skull clamp (300). As shown, flex coil (100) can be positioned and flexibly oriented such that it would be beneath a patient's head if the patient were stabilized using skull clamp (300). Also, as shown, flex coil (100) can be flexibly manipulated such that is follows the general contour of the patient's head (or other body part of interest in other versions). For example, a patient may be lying on their back with their nose facing away from flex coil (100). The patient may be stabilized with skull clamp (300) by tightening first and second pin assemblies (306, 308) (shown without pins). Drape (200) is then coupled with skull clamp (300). For example, first and second tie straps (220, 222) are selectively attached with first and second arms (302, 304) respectively. In particular, first and second tie straps (220, 222) are tied around an upper portion of first and second arms (302, 304) respectively forming knots. In some versions, first and second tie straps (220, 222) can include an adhesive backing or tape or hook material to facilitate connecting first and second tie straps (220, 222) with portions of skull clamp (300). With the attachment of drape (200) to skull clamp (300), drape (200) can be prevented from unintended movement during a surgical or diagnostic procedure. So, in the present examples, drape (200) is a multi-function drape that protects flex coil (100) while also attaching to a stabilization device, e.g. skull clamp (300), to prevent drape (200) from unintended movement. In this way, drape (200) is secured with the stabilizing device that is configured to stabilize a patient or portion of a patient.

While FIG. 4 depicts first and second tie straps (220, 222) being positioned on outer regions of first portion (202) for attachment to upper portions of first and second arms (302, 304), in some other versions, first and second tie strap (220, 222) can be located on other regions of drape (200) for subsequent attachment with one or more portions of a head fixation device, e.g. skull clamp (300). In view of the teachings herein, other ways to configure and attach first and second tie straps (220, 222) to a stabilization device of some kind, e.g. skull clamp (300), will be apparent to those of ordinary skill in the art.

In some versions, drape (200) can be a sterile drape that may be sterilized and then packaged in a sterilized packaging before opening for installation and use. However, in some versions where flex coil (100) is placed in the unsterile field, it may not be required that drape (200) is a sterile drape.

In the illustrated versions, drape (200) is constructed from a clear or translucent plastic material. In some other versions, drape (200) may be constructed from an opaque material. By way of example only, and not limitation, in some versions drape (200) may be comprised of a nonwoven fabric. The various components of drape (200) can be comprised of the same material, or in some versions, components of drape (200) can be comprised of different materials. By way of example only, and not limitation, first, second, and third tie straps (220, 222, 224) can be comprised from a woven or nonwoven fabric while first and second portions (202, 204) can be comprised of a clear plastic. In view of the teachings herein, other materials and constructions for drape (200) will be apparent to those of ordinary skill in the art.

Drape (200), in the present example, is disposable such that it is used for a single patient's procedure and then discarded. Of course disposability of drape (200) is not required in all versions. In versions where drape (200) may be reusable, drape (200) may be sterilizable, e.g., via radiation or another sterilization technique.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometries, materials, dimensions, ratios, steps, and the like discussed above are illustrative and are not required. Accordingly, the scope of the present invention should be considered in terms of any claims that may be presented and is understood not to be limited to the details of structure and operation shown and described in the specification and drawings.

I/we claim:

1. A drape for use in a surgical or diagnostic procedure using MRI and a patient stabilization device, wherein the drape is configured to protect a coil portion of a coil, the drape comprising:
(a) a first portion that covers the coil portion; and
(b) one or more attachment members that are configured to selectively attach the drape to the stabilization device.

2. The drape of claim 1, wherein the drape provides complete coverage of the coil portion.

3. The drape of claim 1, wherein the one or more attachment members are configured to selectively attach the drape to a skull clamp.

4. The drape of claim 1, wherein the drape comprises a second portion having a smaller size than the first portion, wherein the second portion connects with the first portion of the drape.

5. The drape of claim 4, wherein the second portion of the drape is configured to cover a portion of the coil separate from the coil portion.

6. The drape of claim 1, wherein the drape comprises a first opening on a first end of the drape and a second opening on a second end of the drape.

7. The drape of claim 6, wherein drape comprises an interior space extending from the first end of the drape to the second end of the drape, wherein the drape is configured for the coil to be insertable through the first opening of the drape.

8. The drape of claim 1, wherein the drape comprises at least one fastener configured to close the first opening of the drape.

9. The drape of claim 1, wherein the drape comprises at least one fastener configured to close the second opening of the drape.

10. The drape of claim 9, wherein the fastener comprises a tape.

11. The drape of claim 1, wherein first portion of the drape is configured to extend past the coil portion when the coil is fully inserted within the drape.

12. The drape of claim 11, wherein the first portion comprises a first surface having a first section and a second section, wherein the first section is configured to fold onto the second section of the first surface.

13. The drape of claim 12, wherein the first portion comprises at least one fastener configured to selectively attach the first section with the second section.

14. The drape of claim 13, wherein the at least one fastener comprises a tape.

15. The drape of claim 1, wherein the one or more attachment members comprise a strap.

16. The drape of claim 15, wherein the one or more attachment members are configured to selectively tie the drape to the stabilization device.

17. The drape of claim 1, wherein the drape is translucent.

18. The drape of claim 1, wherein the drape accommodates a flexible coil portion of a coil.

19. A drape for use in a surgical or diagnostic procedure using MRI and a stabilization device, wherein the stabilization device contacts a portion of a patient to stabilize the patient, wherein the drape is configured to protect a portion of a MRI coil, the drape comprising:
(a) an surface configured to cover the portion of the MRI coil; (b) at least one fastener configured to selectively attach the drape with the portion of the MRI coil; and
(c) at least one attachment member configured to selectively attach the drape to the stabilization device.

20. A method of using a drape in a surgical or diagnostic procedure using MRI and a patient stabilization device, the method comprising the steps of:
(a) covering a portion of a MRI coil with the drape;
(b) securing the drape with the portion of the MRI coil to inhibit movement of the drape relative to the portion of the MRI coil; and
(c) attaching the drape directly to the stabilization device to inhibit movement of the drape relative to the stabilization device.
